(19) United States
(12) Patent Application Publication
(10) Pub. No.: US 2018/0143177 A1
(43) Pub. Date: May 24, 2018

(54) PROCESS COMPRISING ANALYSING A FLOWING FLUID

(71) Applicant: SHELL OIL COMPANY, HOUSTON, TX (US)

(72) Inventor: Cornelis Pieter Wilhelmus DE GRAAF, Amsterdam (NL)

(21) Appl. No.: 15/572,848

(22) PCT Filed: May 4, 2016
(86) PCT No.: PCT/EP2016/059978

§ 371 Date: Nov. 9, 2017

(30) Foreign Application Priority Data
May 12, 2015 (EP) 15167307.6

(51) Int. Cl. G01N 33/28

(52) U.S. Cl. CPC G01N 33/2841; G01N 33/2823

(57) ABSTRACT

A process which comprises (a) injecting stripping gas into the fluid flow, (b) taking a sample from the stripping gas containing fluid obtained in step (a) at a point downstream of the injection of the stripping gas, and (c) analyzing the sample taken in step (b).

### FIELD OF THE INVENTION

The present invention relates to a process more specifically a process for analysis of a fluid flow comprising hydrocarbon oil.

### BACKGROUND OF THE INVENTION

In the recovery of oil from a subterranean formation, only a portion of the oil in the formation generally is recovered using primary recovery methods utilizing the natural formation pressure to produce the oil. A portion of the oil that cannot be produced from the formation using primary recovery methods may be produced by secondary recovery methods such as water flooding. Oil that cannot be produced from the formation using primary recovery methods and optionally secondary methods such as water flooding, may be produced by so-called enhanced oil recovery methods. These methods also are referred to as EOR methods.

Chemical enhanced oil recovery can utilize recovering agent, surfactant or polymer or a combination of one or more of these compounds with other chemicals such as polymer and/or gas to flood an oil-bearing formation to increase the amount of oil recovered from the formation. Recovering agents are well known and can be miscible or immiscible with the oil.

The mixture which is produced from an oil-bearing formation with the help of a recovering agent generally contains oil and water and also the recovering agent itself after the recovering agent has been injected for some time. It can be necessary to detect when the recovering agent starts to show up in the recovered fluid (“breaks through”) for example to start recovery of the recovering agent from the fluid.

Furthermore, it can be crucial to monitor whether certain contaminants or hazardous components such as hydrogen sulphide are present in fluid which is recovered from an oil containing formation.

We now have found a process which allows to analyze the composition of a fluid flow in an efficient way.

### SUMMARY OF THE INVENTION

The present invention relates to a process comprising the steps of (a) injecting stripping gas into a fluid flow comprising hydrocarbon oil, (b) taking a sample from the stripping gas containing fluid obtained in step (a) at a point downstream the injection of the stripping gas, and (c) analyzing the sample taken in step (b).

### BRIEF DESCRIPTION OF THE DRAWINGS

FIG. 1 shows an embodiment of the process of the present invention in detail and by way of example only. This process applies a static mixer to mix the stripping gas with the fluid flow.

### DETAILED DESCRIPTION OF THE INVENTION

An advantage of the process of the present invention is that it does not make use of valves and therefore can be carried out continuously without wear and tear. Another advantage is that it is simple and reliable and only requires limited changes to the equipment already in place.

Preferably, the analysis of step (c) comprises detecting a specific compound or group of compounds in the fluid flow. It is preferred to analyse the sample for a single compound or a specific group of compounds only because this makes that no full analysis of the sample needs to be carried out. The analysis can consist of determining whether the specific compound or group of compounds is present. It is preferred that the compound or compounds of which the presence is to be determined is a gas at the conditions at which the stripping gas is introduced into the fluid flow.

The fluid flow can be any mixture comprising hydrocarbon oil, more specifically hydrocarbon mineral oil, such as mixtures obtained in the recovery of oil from an oil-bearing formation. A hydrocarbon oil is a liquid containing compounds comprising carbon and hydrogen. It is possible to apply the process of the present invention to the main flow of the fluid recovered from the formation. However, the volume of such main flow will generally be large which means that a large amount of stripping gas would have to be injected for the present process. Therefore, it will often be preferred to take a sample from a fluid flow line which contains fluid which is separated from the main flow of fluid.

The present invention is especially suitable for analyzing a fluid flow comprising both gas and liquid and more preferably a fluid flow which is obtained in recovering oil from an oil-bearing formation. Oil containing fluids which have been recovered from an oil-bearing formation, will contain crude oil and optionally further compounds present in the formation. Furthermore, the fluid generally will contain water. Water can originate from aqueous recovery solution which has been injected or from the formation itself. In addition, the fluid to be analysed can contain gaseous hydrocarbons and/or hydrogen sulphide which either originate from the formation or are formed during recovery of the oil. It is especially advantageous to be able to detect hydrogen sulphide. The present process would be suitable for such detection.

Oil may be recovered from an oil-bearing formation with the help of a recovering agent. Many suitable recovering agents are known such as dimethyl ether, methyl tertiary butyl ether (MTBE), ethyl tertiary butyl ether (ETBE), tertiary amyl methyl ether (TAME), dimethoxy methane, mono-ethylene glycol, di-ethylene glycol, diethyl ether, carbon disulphide and dimethyl sulphide. Preferably, the agent is selected from the group consisting of dimethyl ether, carbon disulphide and dimethyl sulphide.

The stripping gas preferably does not chemically react with a compound in the fluid. Therefore, the stripping gas preferably is an inert gas. Many inert gases are known and available. Nitrogen is a preferred stripping gas because it is inert and readily available.

The stripping gas preferably is injected into the flow of the fluid at a pressure which is higher than the pressure of the fluid flow in order for mixing to take place. Preferably, the pressure difference is at least 1 bar, more preferably at least 2 bar, most preferably at least 5 bar. The pressure difference preferably is at most 10 bar.

The stripping gas preferably has a temperature of from 0 to 300° C., more preferably of from 10 to 250° C., more preferably at least 50° C. and more preferably at most 200° C. The hydrocarbon fluid flow which is contacted with the stripping gas preferably has a temperature of from 0 to 300° C., more preferably of from 10 to 250° C., more preferably at least 50° C. and more preferably at most 200° C.

Any equipment known to be suitable can be used for injecting the stripping gas into the fluid flow. To further enhance mixing of the stripping gas and the fluid, a static mixer can be present downstream from the injection of the stripping gas and upstream of taking the sample. Therefore, it is preferred that step (a) comprises injecting stripping gas into the fluid flow and subsequently mixing the stripping gas and fluid with the help of a static mixer before step (b).

The stripping gas is added to the flow of hydrocarbon fluid before a sample is taken. This makes that the sample which is removed and analyzed in step (b) will contain both stripping gas and fluid. As the latter generally will be a mixture of gas and liquid, the sample generally will contain gas and liquid. The analysis of step (c) will have to be appropriate for the combination of stripping gas and hydrocarbon fluid in question. Alternatively, the fluid consists mainly of gas in which case the sample can be gaseous.

As the stripping gas generally will not react with a compound in the fluid, the sample can be taken from the mixture of stripping gas and fluid at any time after injecting the stripping gas. However, it generally is preferred not to have too much time lapse between injecting the stripping gas and taking the sample. Therefore, the distance between injecting stripping gas and taking a sample preferably is of from 0.01 to 0.50 m, more specifically of from 0.1 to 0.4 m.

The sample can be taken in any way known to somebody skilled in the art. A simple and suitable method consists of a tube connected to and in fluid communication with the tube through which the fluid flows. The tube used for taking the sample preferably has a smaller diameter than the tube for the main flow of fluid. Preferably, the sample is taken with the help of a tube having upstream of the actual analysis a structured packing and/or membrane. A structured packing can be used to prevent froth. A membrane can be used to ensure that only a limited amount of fluid and most preferably gas is removed for analysis. A preferred sample is permeate separated with the help of a membrane from the stripping gas containing fluid obtained in step (a). Specific compounds can be separated from the fluid by use of an appropriate membrane so that a first separation already has taken place and the analysis is even easier to carry out. A further advantage is that less fluid needs to be removed and possibly is wasted.

The analysis of the compounds present in the sample can be carried out in any way known to be suitable. In many cases, gas chromatography is a suitable method for detecting a specific compound or group of compounds.

After analysis, any remaining sample can be combined again with the fluid flow. Preferably, the remaining sample is again combined with the main fluid flow downstream from where the original sampling took place.

The process of the present invention is especially suitable for continuously taking samples as no moving parts are involved. Furthermore, the fluid flow can be analyzed at high frequency or continuously for the presence of compounds such as hydrogen sulphide and/or recovering agent. This allows analysis of a fluid flow at least every day, more specifically at least every hour.

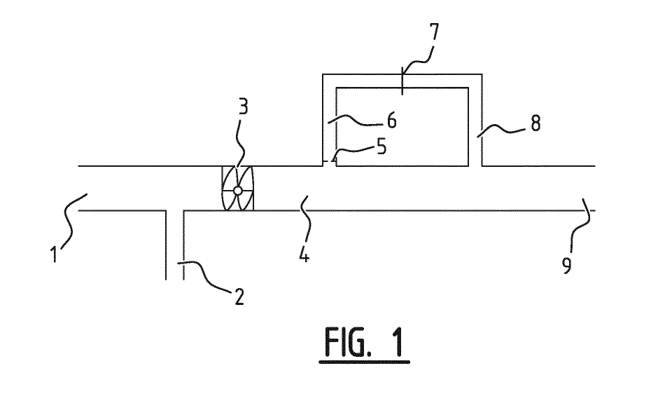

An embodiment of the process of the present invention is shown in FIG. 1.

FIG. 1 shows a line-up of a process according to the invention in which a static mixer is applied to mix the stripping gas with the fluid flow and a membrane is used in taking a sample.

A specific embodiment of the invention is shown herein by way of example only. The invention is susceptible to various modifications. It should be understood that the process shown is not intended to limit the invention to the particular process disclosed but on the contrary, the intention is to cover all modifications, equivalents and alternatives falling within the scope of the invention as defined by the appended claims.

In the process of FIG. 1, a fluid comprising oil and water is recovered from an oil-bearing formation and flows from left to right via tube 1. Via tube 2 stripping gas is injected which stripping gas is thoroughly mixed with the fluid with the help of static mixer 3. The mixture thus obtained continues flowing through tube 2 while continuously a small amount of the fluid containing stripping gas is removed from the main flow of fluid 4 via tube 6. A limited amount of fluid containing inert gas is removed via tube 6 via membrane 5. Permeate obtained at the downstream side of membrane 5 flows via line 6 to an analyzer 7. The analyzer 7 can be any equipment known to be suitable by the person skilled in the art. Preferably, the analysis is carried out with the help of a suitably calibrated gas chromatograph. The sample to be analyzed can be removed for analysis or analysis can be carried out on the fluid flowing through tube 6. Any sample which is left after analysis can be sent back via tube 8 to the main fluid flow in tube 9.

1. A process which comprises
(a) injecting stripping gas into a fluid flow comprising hydrocarbon oil,
(b) taking a sample from the stripping gas containing fluid obtained in step (a) at a point downstream of the injection of the stripping gas, and
(c) analyzing the sample taken in step (b).

2. The process according to claim 1, in which the fluid flow is obtained in recovering oil from an oil-bearing formation.

3. The process according to claim 2, in which process oil is recovered with the help of a recovering agent, preferably an agent selected from the group consisting of carbon disulfide, dimethyl sulfide and dimethyl ether.

4. The process according to claim 1 in which process the stripping gas is nitrogen.

5. The process according to claim 1 in which process the distance between injecting stripping gas and taking a sample is of from 1 to 50 cm.

6. The process according to claim 1 in which process step (a) comprises injecting stripping gas into the fluid flow and subsequently mixing the stripping gas and fluid with the help of a static mixer before step (b).

7. The process according to claim 1 in which the sample of step (b) is permeate separated from the stripping gas containing fluid obtained in step (a) with the help of a membrane.

8. The process according to claim 1, in which sample taken in step (b) subsequently is combined again with the fluid flow.

9. The process according to claim 1, in which process the sample is taken continuously.

10. The process according to claim 3, in which process the fluid flow is analyzed for the presence of the recovering agent.
